(19) United States
(12) Patent Application Publication
(10) Pub. No.: US 2018/0155730 A1
(43) Pub. Date: Jun. 7, 2018

(54) Recombinant Microorganism Including Genetic Modification That Increases Activity Of Haloalkane Dehalogenase And Genetic Modification That Increases Expression Of At Least One Of Rclr, Rcla, Rclb, And Rclc, And Method Of Reducing Concentration Of Fluorinated Methane In Sample

(71) Applicants: Samsung Electronics Co., Ltd., Suwon-si (KR); Korea Advanced Institute of Science and Technology, Daejeon (KR)

(72) Inventors: Yukyung Jung, Hwaseong-si (KR); Byungkwan Cho, Daejeon (KR); Donghui Choe, Chilgok-gun (KR); Taeyong Kim, Daejeon (KR); Jinhwan Park, Suwon-si (KR)

(21) Appl. No.: 15/828,746

(22) Filed: Dec. 1, 2017

(30) Foreign Application Priority Data
Dec. 1, 2016 (KR) 10-2016-0162911

(51) Int. Cl. C12N 15/70; C12N 1/20; C07C 19/08

(52) U.S. Cl. CPC C12N 15/70; C07C 19/08; C12Y 308/01005; C12N 1/20

(57) ABSTRACT

Provided are a recombinant microorganism including a first genetic modification that increases activity of haloalkane dehalogenase (HAD) and a second genetic modification that increases expression of at least one of RcIR, RcIA, RcIB, and RcIC; a composition for reducing a concentration of fluorinated methane in a sample, wherein the composition includes the recombinant microorganism; and a method of reducing a concentration of fluorinated methane in a sample.

### CROSS-REFERENCE TO RELATED APPLICATION

This application claims the benefit of Korean Patent Application No. 10-2016-0162911, filed on Dec. 1, 2016, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 19,439 Byte ASCII (Text) file named “736174_ST25.TXT,” created on Nov. 30, 2017.

### BACKGROUND

### 1. Field

The present disclosure relates to a recombinant microorganism including a first genetic modification that increases activity of haloalkane dehalogenase (HAD) and a second genetic modification that increases expression of at least one of RcIR, RcIA, RcIB, and RcIC, a composition for reducing a concentration of fluorinated methane in a sample, wherein the composition includes the recombinant microorganism, and a method of reducing a concentration of fluorinated methane in a sample.

### 2. Description of the Related Art

One of the most serious environmental problems is the emission of greenhouse gases which accelerate global warming, and regulations aimed at reducing and preventing the emission of greenhouse gases have been tightened. Among the greenhouse gases, fluorinated gases (F-gases) such as perfluorocarbons (PFCs), hydrofluorocarbons (HFCs), and sulfur hexafluoride (SF₆) show low absolute emission but have a long half-life and a very high global warming potential, resulting in significant adverse environmental impacts. The amount of F-gases emitted from semiconductor and electronics industries, which are major sources of F-gas emission, has exceeded the assigned limits of greenhouse gas emissions and continues to increase. Therefore, the costs required to degrade greenhouse gases and costs required to meet greenhouse gas emission allowances are increasing every year.

A pyrolysis or catalytic thermal oxidation process has generally been used for the decomposition of F-gases. However, this process has disadvantages in terms of limited decomposition rate, emission of secondary pollutants, and high cost. To help solve these problems, biological decomposition of F-gas using a microbial biocatalyst has been adopted. Accordingly, this approach is expected to overcome the limitations of the chemical decomposition process and allow F-gases to be treated in a more economical and environmentally friendly manner.

Therefore, there is a need to develop new microorganisms and methods for the biological decomposition of F-gases. This invention provides such microorganisms and methods.

### SUMMARY

Provided is a recombinant microorganism including a first genetic modification that increases activity of haloalkane dehalogenase (HAD) and a second genetic modification that increases expression of at least one selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4.

Provided is a composition for reducing a concentration of fluorinated methane represented by CHₙF₄₋ₙ (wherein n is an integer from 0 to 3) in a sample, wherein the composition includes a recombinant microorganism including a first genetic modification that increases activity of HAD and a second genetic modification that increases expression of at least one selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4.

Provided is a method of reducing a concentration of fluorinated methane represented by CHₙF₄₋ₙ (wherein n is an integer from 0 to 3) in a sample, wherein the method includes contacting a recombinant microorganism with the sample including fluorinated methane, and the recombinant microorganism includes a first genetic modification that increases activity of HAD and a second genetic modification that increases expression of at least one selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates a pETD-dhIA vector; and

FIGS. 2 to 4 respectively illustrate a pACYCDuet-RcIR vector, a pACYCDuet-RcIA vector, and a pACYCDuet-RcIABC vector.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. The term “increase in activity”, or “increased activity” or the like, as used herein, may refer to a detectable increase in the activity of a cell, a polypeptide, a protein, or an enzyme. The term “increase in activity” or “increased activity” or the like may refer to an activity level of a modified (e.g., genetically engineered) cell, protein, or enzyme that is higher than that of a comparable cell, protein, or enzyme of the same type, such as a cell, protein, or enzyme that does not have a given genetic modification (e.g., the original or “wild-type” cell, polypeptide, protein, or enzyme). The term “activity of a cell” may refer to activity of a specific protein or enzyme of the cell. For example, an activity level of the modified or engineered cell, protein, or enzyme may be increased by about 5% or greater, about 10% or greater, about 15% or greater, about 20% or greater, about 30% or greater, about 50% or greater, about 60% or greater, about 70% or greater, or about 100% or greater, as compared with that of an unmodified cell, protein, or enzyme of the same type, e.g., a wild-type cell, protein, or enzyme. An activity level of a specific protein or enzyme of a cell may be increased by about 5% or greater, about 10% or greater, about 15% or greater, about 20% or greater, about 30% or greater, about 50% or greater, about 60% or greater, about 70% or greater, or about 100% or greater, as compared with that of the same protein or enzyme of a parent cell, e.g., an unmodified cell. An increase in activity of a protein or enzyme may be measured by using a method known to those of ordinary skill in the art.

An increase in activity of an enzyme or polypeptide may be achieved by an increase in the expression or specific activity thereof. The increase in the expression may be achieved by introduction of a polynucleotide encoding the enzyme or polypeptide into a cell (e.g., an increase in a copy number), or by a mutation in the regulatory region of the polynucleotide. The polynucleotide encoding the enzyme or polypeptide may be operably linked to a regulatory sequence that allows expression thereof, for example, a promoter, an enhancer, a polyadenylation region, or a combination thereof. The polynucleotide which is introduced externally or whose copy number is increased may be endogenous or heterologous. The term “endogenous gene” refers to a gene which is included in a microorganism prior to introducing the genetic modification (e.g., native gene). The term “heterologous” refers to a gene that is “foreign,” or “not native” to the species. In either case, a polynucleotide or gene that is externally introduced into a cell is referred to as “exogenous,” and an exogenous gene or polynucleotide may be homologous or heterologous with respect to a host cell into which the gene is introduced. Thus, the microorganism into which the polynucleotide encoding the enzyme is introduced may be a microorganism that already includes the gene encoded by the polynucleotide (e.g., the gene or polynucleotide is endogenous to the microorganism). Alternatively, the microorganism can be without a copy of the gene prior to its introduction (e.g., the polynucleotide or gene is heterologous to the microorganism)

The term “increase in expression” or “overexpression”, as used herein, refers to a genetically modified cell that has a detectable increase in the level of a specific expression product, as compared with a comparative cell, e.g., a cell of the same type without the genetic modification (e.g., a native or wild-type cell). For example, the increase may be by about 5% or greater, about 10% or greater, about 15% or greater, about 20% or greater, about 30% or greater, about 50% or greater, about 60% or greater, about 70% or greater, or about 100% or greater, as compared with a cell without the genetic modification. The increase in expression may be caused by an increase in the copy number of a gene, an increase in transcription, or an increase in translation.

The increase in expression may be identified by any suitable method known in the art. For example, the increase in expression may be measured by immunohistochemistry using anti-expression product antibodies, a diagnostic assay such as fluorescence-activated cell sorting (FACS) analysis, or a prognostic assay. Alternatively, a level of a nucleic acid encoding an expression product or of mRNA in a cell may be measured in the cell by fluorescence in situ hybridization (FISH) using a nucleic acid encoding the expression product or a complementary nucleic acid probe thereof, Southern blotting, Northern blotting, or a polymerase chain reaction (PCR) such as real-time quantitative PCR. Alternatively, the increase in expression may be determined by measuring a level of a downstream expression product of an expression product such as RcIR, for example, a level of RcIA, RcIB, RcIC, or a combination thereof. The term “downstream expression product” refers to an expression product of a gene whose expression is regulated by RcIR, which is a transcription factor.

The “increase in the copy number” of a gene may be caused by amplification of a gene already existing in the microorganism or an introduction of an exogenous gene. An increase in copy number encompasses the introduction of an exogenous gene that does not exist in a non-engineered cell (i.e., prior to the introduction of the exogenous gene). The introduction of the gene may be mediated by a vehicle such as a vector. The introduction may be a transient introduction in which the gene is not integrated into the genome, or may be an introduction that results in integration of the gene into the genome. The introduction may be performed, for example, by introducing a vector into the cell, in which the vector includes a polynucleotide encoding a target polypeptide and replicating the vector in the cell; or by integrating the polynucleotide into the genome.

The introduction of the gene may be performed by any known method, such as transformation, transfection, and electroporation. The gene may be introduced via a vehicle, or may be introduced by itself. The term “vehicle” as used herein refers to a nucleic acid molecule that is capable of delivering other nucleic acids linked thereto. As a nucleic acid sequence mediating introduction of a specific gene, the vehicle as used herein is construed to be interchangeable with a nucleic acid construct and a cassette. The vehicle may include a vector. Examples of the vector include a plasmid, a virus-derived vector, or the like. A plasmid is a circular double-stranded DNA molecule linkable with another DNA. Examples of the vector include a plasmid expression vector and a virus expression vector, e.g., a replication-defective retrovirus, adenovirus, adeno-associated virus, and a combination thereof.

The term “parent cell” refers to an original cell, e.g., a non-genetically engineered cell of the same type as the engineered microorganism. With regard to a specific genetic modification, the parent cell may be a cell that lacks the specific genetic modification, but is identical in all other respects. Thus, the parent cell may be a cell that is used as a starting material to produce a genetically engineered cell having an increased activity or expression of a given protein (e.g., a protein having about 95% or greater sequence identity to HAD). The same comparison applies to different genetic modifications.

The term “gene” as used herein refers to a nucleic acid fragment expressing a specific protein. A gene may include regulatory sequences such as a 5′ non-coding sequence and/or a 3′ non-coding sequence, or may be free of regulator sequences.

The term “sequence identity” of a nucleic acid or polypeptide, as used herein, refers to a degree of identity between nucleotide bases or amino acid residues of sequences obtained after the sequences are aligned so as to best match in specific comparable regions. The sequence identity is a value measured by comparing two sequences in specific comparable regions via optimal alignment of the two sequences, in which portions of the sequences in the specific comparable regions may be added or deleted compared to reference sequences. A percentage of sequence identity may be calculated by, for example, comparing two optimally aligned sequences in the entire comparable regions, determining the number of locations in which the same amino acids or nucleotides appear to obtain the number of matching locations, dividing the number of matching locations by the total number of locations in the comparable regions (that is, the size of a range), and multiplying the result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), MegAlign™ (DNASTAR Inc), etc.

The term “genetic modification” as used herein includes artificial alteration in a constitution or structure of genetic materials of a cell.

Unless stated otherwise, percent composition (%) is expressed as w/w %.

An aspect of the present disclosure provides a recombinant microorganism including a first genetic modification that increases activity of haloalkane dehalogenase (HAD) and a second genetic modification that increases expression of at least one gene selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4.

HAD is an enzyme that catalyzes the reaction of 1-haloalkane and water as substrates to produce primary alcohol and halide. This enzyme belongs to the family of hydrolases acting on halide bonds in carbon-halide compounds. However, the recombinant microorganism is not necessarily construed as being limited to this specific mechanism in reducing a concentration of fluorinated methane represented by CHₙF₄₋ₙ (wherein n may be an integer from 0 to 3) in a sample. The HAD may be classified as EC 3.8.1.5. The HAD may be selected from the group consisting of HADs from the genus Xanthobacter, the genus Rhodococcus, the genus Sphingomonas, the genus Bacillus, the genus Pseudomonas, the genus Azotobacter, the genus Agrobacterium, and the genus Escherichia. The HAD may be from X. autotrophicus. The HAD may have about 90% or greater, about 95% or greater, about 96% or greater, about 97% or greater, about 98% or greater, or about 99% or greater sequence identity to an amino acid sequence of SEQ ID NO: 11. The HAD may have an amino acid sequence of SEQ ID NO: 11. At least one exogenous gene encoding the HAD may have a nucleotide sequence of SEQ ID NO: 9 or 10. The gene may be codon-optimized with respect to the recombinant microorganism acting as a host cell. Codon optimization refers to production of a gene in which one or more endogenous codons are replaced with codons for the same amino acid but of preference in the corresponding host. The nucleotide sequence of SEQ ID NO: 9 is a gene encoding a haloalkane dehalogenase (dhIA) derived from X. autotrophicus.

The RcIR protein is a transcription factor activated in E. coli by reactive chlorine species (RCS) such as hypochlorous acid (HOCl). RcIR may include conserved cysteine residues that are specifically sensitive to oxidation by RCS. The conserved cysteine residues may be Cys-21 and Cys-89. Reversible oxidation of conserved cysteine residues may activate RcIR to control expression of a gene essential for survival of HOCl stress. The gene essential for survival may be RcIA, RcIB, RcIC, or a combination thereof of which expression is activated by RcIR. RcIA may be probable pyridine nucleotide-disulfide oxidoreductase RcIA. The function of RcIB has yet to be verified. RcIC may be an inner membrane protein.

In the recombinant microorganisms described herein, the RcIR gene, the RcIA gene, the RcIB gene, and the RcIC gene may be from a microorganism selected from the group consisting of the genus Xanthobacter, the genus Bacillus, the genus Pseudomonas, the genus Azotobacter, the genus Agrobacterium, and the genus Escherichia. The RcIR gene, the RcIA gene, the RcIB gene, and the RcIC gene may be derived from Escherichia coli (E. coli). The RcIR protein, the RcIA protein, the RcIB protein, and the RcIC protein may each have about 90% or greater, 95% or greater, 96% or greater, 97% or greater, 98% or greater, or 99% or greater sequence identity to amino acid sequences of SEQ ID NO: 1, SEQ ID NO: 2, SEQ ID NO: 3, and SEQ ID NO: 4, respectively. The RcIR gene, the RcIA gene, the RcIB gene, and the RcIC gene may each have about 90% or greater, 95% or greater, 96% or greater, 97% or greater, 98% or greater, or 99% or greater sequence identity to nucleotide sequences of SEQ ID NO: 5, SEQ ID NO: 6, SEQ ID NO: 7, and SEQ ID NO: 4, respectively.

In an aspect of the invention, the first genetic modification may increase a copy number of a gene encoding HAD, and the second genetic modification may increase a copy number of at least one selected from the group consisting of the gene encoding an RcIR protein having about 95% or greater sequence identity to an amino acid sequence of SEQ ID NO: 1; the gene encoding an RcIA protein having about 95% or greater sequence identity to an amino acid sequence of SEQ ID NO: 2; the gene encoding an RcIB protein having about 95% or greater sequence identity to an amino acid sequence of SEQ ID NO: 3; and the gene encoding an RcIC protein having about 95% or greater sequence identity to an amino acid sequence of SEQ ID NO: 4.

The increase in copy number can be facilitated, for instance, by introducing one or more exogenous nucleic acids encoding the genes. Furthermore, Multiple copies of the foregoing genes may be introduced, for instance, 2 or greater, 5 or greater, 10 or greater, 30 or greater, 50 or greater, 100 or greater, or 1,000 or greater copies of the gene(s). Multiple separate nucleic acids (e.g., vectors) can be used, or multiple genes (the same or different) can be encoded by a single nucleic acid (e.g., vector).

The recombinant microorganism may include a first genetic modification that increases activity of HAD and a second genetic modification that may increase expression of the gene encoding an RcIR protein having about 95% or greater sequence identity to an amino acid sequence of SEQ ID NO: 1; the gene encoding an RcIA protein having about 95% or greater sequence identity to an amino acid sequence of SEQ ID NO: 2; the gene encoding an RcIB protein having about 95% or greater sequence identity to an amino acid sequence of SEQ ID NO: 3; and the gene encoding an RcIC protein having about 95% or greater sequence identity to an amino acid sequence of SEQ ID NO: 4. The recombinant microorganism may include a first genetic modification that increases activity of HAD and a second genetic modification that increases expression of the gene encoding an RcIR protein having about 95% or greater sequence identity to an amino acid sequence of SEQ ID NO: 1; or a first genetic modification that increases activity of HAD and a second genetic modification that increases expression of the gene encoding an RcIA protein having about 95% or greater sequence identity to an amino acid sequence of SEQ ID NO: 2; or a first genetic modification that increases activity of HAD and a second genetic modification that increases expression of the gene encoding an RcIA protein having about 95% or greater sequence identity to an amino acid sequence of SEQ ID NO: 2, and the gene encoding an RcIB protein having about 95% or greater sequence identity to an amino acid sequence of SEQ ID NO: 3, and the gene encoding an RcIC protein having about 95% or greater sequence identity to an amino acid sequence of SEQ ID NO: 4.

The recombinant microorganism may belong to the genus Escherichia, the genus Xanthobacter, the genus Rhodococcus, the genus Sphingomonas, the genus Bacillus, the genus Pseudomonas, the genus Azotobacter, or the genus Agrobacterium. The recombinant microorganism may be E. coli or Xanthobacter autotrophicus.

The recombinant microorganism may be capable of removing increased amounts of fluorinated methane from a sample as compared with a parent strain thereof, wherein the fluorinated methane is represented by CHₙF₄₋ₙ (wherein n may be an integer from 0 to 3).

The recombinant microorganism may reduce a concentration of CHₙF₄₋ₙ (wherein n may be an integer from 0 to 3) in a sample. The fluorinated methane may be reduced by introducing a hydroxyl group to carbon of the fluorinated methane by action of the protein on the C—F or C—H bond thereof or by accumulating the fluorinated methane inside the cell of the microorganism. Further, the fluorinated methane may be reduced by cleaving of the C—F bond of the fluorinated methane, by converting the fluorinated methane into other materials, or by intracellular accumulation of the fluorinated methane. The sample may be in a liquid or gas state. The sample may be industrial waste water or waste gas. The sample may be any material that includes the fluorinated methane. The fluorinated methane may be CF₄, CHF₃, CH₂F₂, CH₃F, or a mixture thereof.

Another aspect of the present disclosure provides a composition for reducing a concentration of fluorinated methane represented by CHₙF₄₋ₙ (wherein n may be an integer from 0 to 3) in a sample comprising any of the recombinant microorganisms described herein.

The composition may be used to reduce the concentration of fluorinated methane in the sample by contacting the composition with the sample. The contacting may be performed in a liquid or solid phase. The contacting may be performed, for example, by contacting a culture medium in which the recombinant microorganism is being cultured with the sample. The culturing may be performed under conditions in which the recombinant microorganism may be allowed to proliferate. The contacting may be performed in a sealed container. The sealed chamber refers to an air tight seal. The contacting may include culturing or incubating the recombinant microorganism while contacting the recombinant microorganism with the sample including fluorinated methane. The contacting may include culturing the recombinant microorganism in a sealed container under conditions in which the recombinant microorganism is allowed to proliferate. The composition may include a medium, or a diluent. The term “medium” refers to a solid, liquid or semi-solid designed to support the growth of the microorganism.

Another aspect of the present disclosure provides a method of reducing a concentration of fluorinated methane represented by CHₙF₄₋ₙ (wherein n is an integer from 0 to 3) in a sample comprising contacting a recombinant microorganism with the sample including fluorinated methane.

The recombinant microorganism for use in the in the inventive method may be any recombinant microorganism or composition described herein. Additionally, the recombinant microorganism for use in the inventive method may be in the form of any of the compositions described herein.

The term “reducing” includes reducing of a concentration of fluorinated methane in the sample by any amount, and includes complete removal of fluorinated methane from the sample. The sample may be a gas or a liquid. The sample may not include the microorganism. The composition may further include a material that increases solubility of the fluorinated methane for a medium or a culture medium.

With regard to the method, the contacting may be performed in a liquid or solid phase. The contacting may be performed, for example, by contacting a culture medium in which the recombinant microorganism is being cultured with the sample. The culturing may be performed under conditions in which the recombinant microorganism may be allowed to proliferate. The contacting may be performed in a sealed container. The contacting may be performed during an exponential phase or a stationary phase of a growth stage of the recombinant microorganism. The culturing may be performed under aerobic or anaerobic conditions. The contacting may be performed in a sealed container under conditions in which the recombinant microorganism may survive or be viable. The conditions in which the recombinant microorganism may survive or be viable may be conditions in which the recombinant microorganism may be allowed to proliferate or remain in a resting state.

With regard to the method, the sample may be in a liquid or gas phase. The sample may be industrial waste water or waste gas. The sample may be passively or actively contacted with the culture of the microorganism. The sample may be, for example, sparged into the culture of the microorganism. That is, the sample may be sparged into a medium or a culture medium. The sparging may be sparging of the sample from the bottom to the top of the medium or the culture medium. The sparging may include injecting of droplets of the sample.

With regard to the method, the contacting may be performed in a batch or continuous manner. The contacting may include, for example, repeatedly contacting a sample with a fresh recombinant microorganism as described herein. Contacting the sample with the fresh recombinant microorganism may be performed twice or more, for example, twice, three times, five times, or ten times or more. The contacting may be continued or repeated until the concentration of fluorinated methane in the sample reaches a desired reduced concentration.

Another aspect of the present disclosure provides a method of producing a microorganism comprising introducing a first genetic modification to the microorganism that increases the activity of haloalkane dehalogenase (HAD) and second genetic modification that increases expression of at least one gene selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4. The microorganism may have improved ability of removing fluorinated methane in a sample.

The method may include introducing genes to the microorganism, wherein the genes may include a gene encoding HAD and at least one gene selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4. The introducing of the genes may be introducing of a vehicle including the genes.

Hereinafter, the inventive concept of the present disclosure will be described in more detail with reference to Examples. However, these Examples are for illustrative purposes only, and the inventive concept is not intended to be limited by these Examples.

### Example 1: Decomposition of Fluorinated Methane by E. coli, in which Haloalkane Dehalogenase (HAD) Gene and at Least One of RcIR Gene, RcIA Gene, RcIB Gene, and RcIC Gene are Introduced into E. coli

(1) Introduction of HAD Gene into E. coli

HAD (dhIA) of Xanthobacter autotrophicus GJ10 was selected as an enzyme having activity of decomposing fluoro-containing hydrocarbon. Xanthobacter autotrophicus GJ10 was purchased from the German Collection of Microorganisms and Cell Cultures (DSMZ).

A gene encoding the HAD (dhIA) amino acid sequence of SEQ ID NO: 11 has a nucleotide sequence of SEQ ID NO: 9; however, in order to optimize the expression thereof in E. coli, a gene having a codon-optimized sequence (SEQ ID NO: 10) was manipulated to have a nucleotide sequence having a high codon usage frequency in E. coli. The gene of SEQ ID NO: 10 was inserted into a pETDuet vector (available from Novagen, Cat. No. 71146-3) which had been cleaved with the restriction enzymes, NcoI and HindIII, by using an InFusion cloning kit (available from Clontech Laboratories, Inc.) to obtain a pETDuet-dhIA vector (hereinafter referred as “pETD-dhIA vector”).

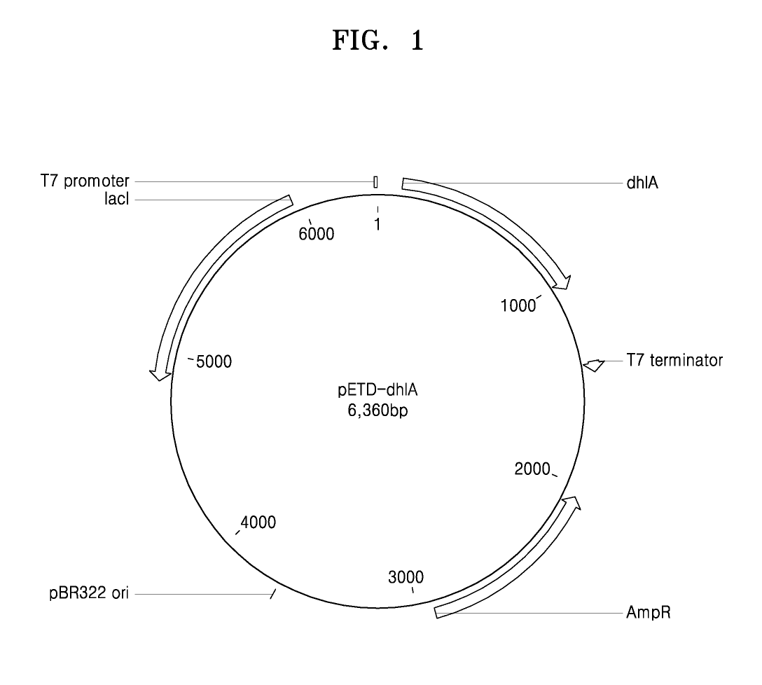

FIG. 1 illustrates the pETD-dhIA vector.

Next, the obtained pETDuet-dhIA vector was introduced into an E. coli BL21 strain by a heat shock method. The E. coli strain was cultured on a Luria-Bertani (“LB”) plate medium including 100 micrograms per milliliter (μg/mL) of ampicillin to select the strain having ampicillin resistance, and the introduction of the pETDuet-dhIA vector was identified by sequencing. Lastly, the HAD-introduced E. coli was designated as ‘BL21/pETduet-dhIA’.

(2) Introduction of at Least One of RcIR Gene, RcIA Gene, RcIB Gene, and RcIC Gene

An RcIR gene; an RcIA gene; and an RcIABC region including an RcIA gene, an RcIB gene, and an RcIC gene, derived from E. coli, were each introduced into the BL21/pETduet-dhIA. The three genes, i.e., the RcIA gene, the RcIB gene, and the RcIC gene, are activated by the RcIR gene, and are known to together constitute a regulon.

From the E. coli BL21 (available from Invitrogen) strain genomic DNA, the RcIR gene, the RcIA gene, and the RcIABC region were amplified. The RcIR, RcIA, RcIB, and RcIC genes respectively have the nucleotide sequences of SEQ ID NOs: 5, 6, 7, and 8. The nucleotide sequences of SEQ ID NOs: 5, 6, 7, and 8 respectively encode the amino acid sequences of SEQ ID NO: 1, 2, 3, and 4. In detail, the E. coli BL21 strain was cultured and stirred at a rate of 230 revolutions per minute (rpm) at a temperature of 37° C. in an LB medium overnight. The genomic DNA was separated by using a total DNA extraction kit (available from Invitrogen Biotechnology). A polymerase chain reaction (PCR) was performed using the separated genomic DNA as a template and a set of primers having nucleotide sequences of SEQ ID NOs: 12 and 13; a set of primers having nucleotide sequences of SEQ ID NOs: 14 and 15; or a set of primers having nucleotide sequences of SEQ ID NOs: 13 and 16 to obtain the amplified RcIR gene, RcIA gene, or RcIABC region, respectively. The amplified RcIR gene, RcIA gene, and RcIABC region were each inserted into a pACYCDuet (available from Novagen, Cat. No. 71147-3) that had been cleaved with the restriction enzymes, NcoI and HindIII, by using the InFusion cloning kit (available from Clontech Laboratories, Inc.) to obtain a pACYCDuet-RcIR vector, a pACYCDuet-RcIA vector, and a pACYCDuet-RcIABC vector, respectively. Hereinafter, the pACYCDuet-RcIR, pACYCDuet-RcIA, and pACYCDuet-RcIABC vectors are also respectively referred as the pAD-RcIR, pAD-RcIA, and pAD-RcIABC vectors.

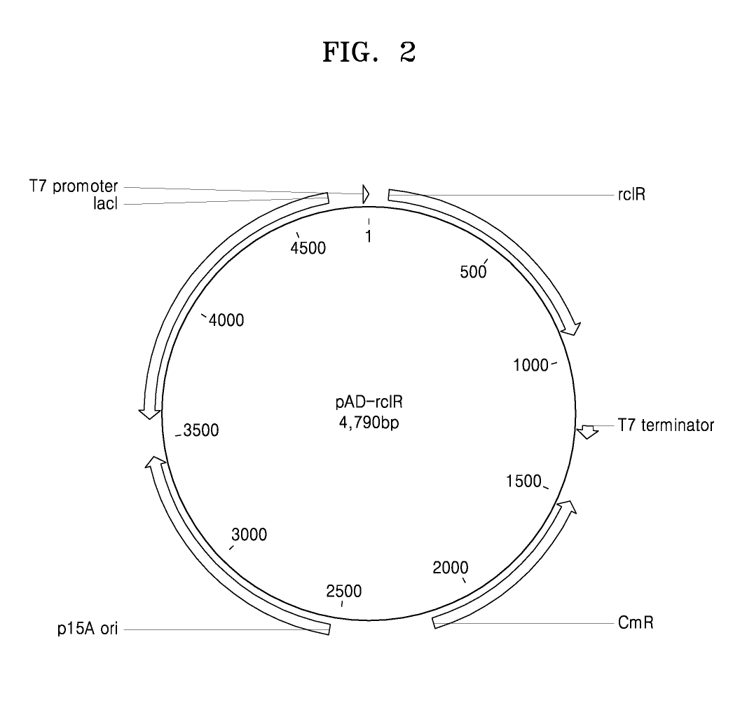

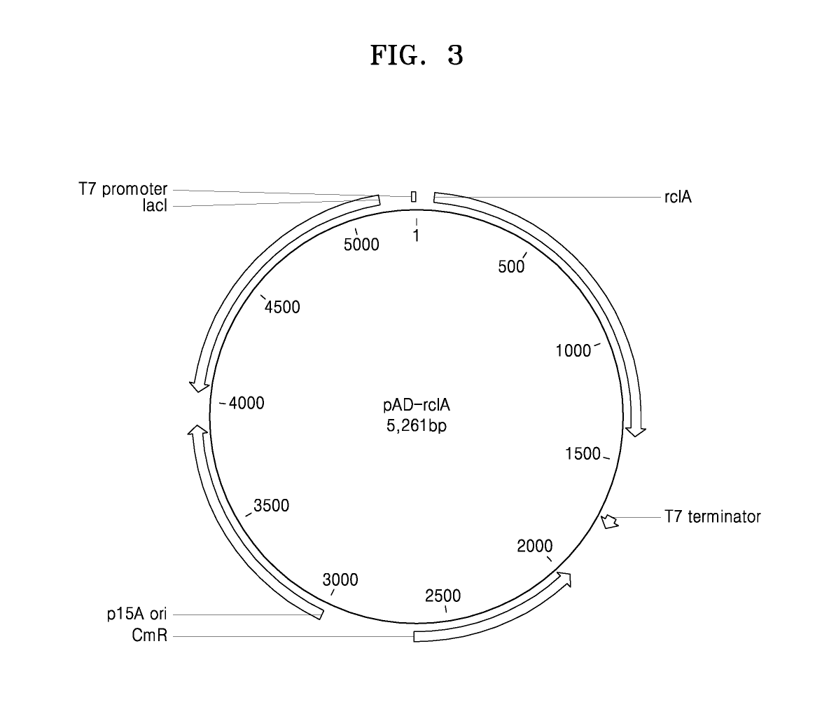

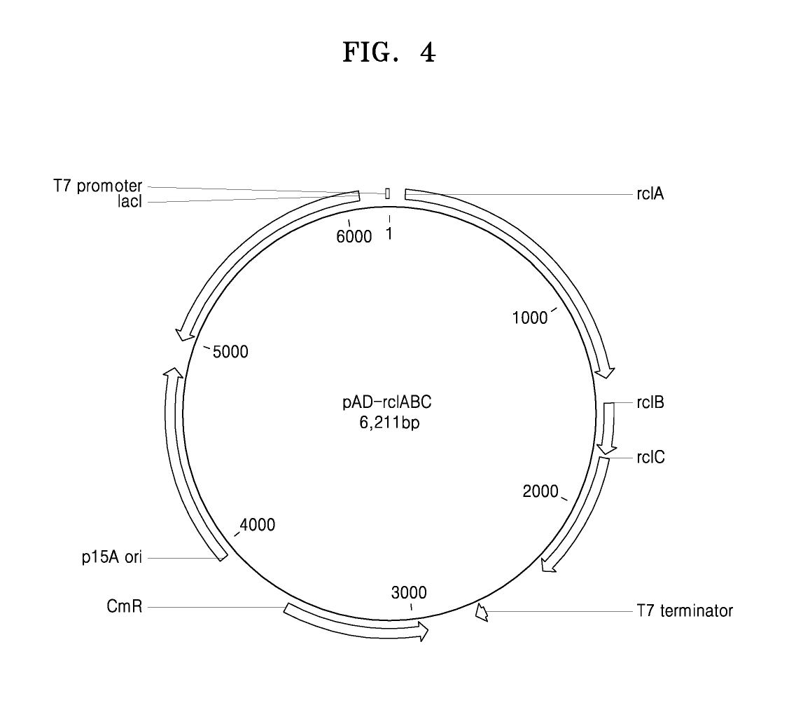

FIGS. 2 to 4 respectively illustrate the pACYCDuet-RcIR vector, the pACYCDuet-RcIA vector, and the pACYCDuet-RcIABC vector.

The pACYCDuet-RcIR, pACYCDuet-RcIA, and pACYCDuet-RcIABC vectors were introduced by a heat shock method into the E. coli BL21/pETduet-dhIA strain obtained in Section (1). The E. coli strains were cultured on an LB plate medium including 100 μg/mL of ampicillin and 35 μg/mL of chloramphenicol to select strains having ampicillin and chloramphenicol resistance. The finally selected strains were designated as E. coli ‘BL21/pETD-dhIA’, ‘BL21/pETD-dhIA+pAD-RcIR’, ‘BL21/pETD-dhIA+pAD-RcIA’, and ‘BL21/pETD-dhIA+pAD-RcIABC’.

(3) Decomposition of Fluorinated Methane by Recombinant E. coli

The E. coli BL21/pETD-dhIA, BL21/pETD-dhIA+pAD-RcIR, BL21/pETD-dhIA+pAD-RcIA, and BL21/pETD-dhIA+pAD-RcIABC obtained in Section (2) were added to a 60 mL serum bottle containing 30 milliliters (mL) of 4 grams per liter (g/L) glucose-containing M9 medium at a concentration of OD₆₀₀=3 and then, CF₄ gas was added in a headspace volume of the serum bottle such that an initial concentration of CF₄ was 1,000 parts per million (ppm). The serum bottle was placed in a shaking incubator (available from Daihan Labtech), and then incubated for 4 days at a temperature of 30° C. while shaking at 230 rpm. Then, the amount of CF₄ in a headspace was analyzed. For analysis, 0.5 mL was collected from the headspace using a syringe and injected into a gas chromatography (GC) (Agilent 7890, Palo Alto, Calif., USA). The injected CF₄ was separated through a CP-PoraBOND Q column (25 m length, 0.32 mm i.d., 5 um film thickness, Agilent), and changes in concentration of the separated CF₄ were analyzed by a Mass Selective Detector (MSD) (Agilent 5973, Palo Alto, Calif., USA). As a carrier gas, helium was applied to the column at a flow rate of 1.5 ml/min. GC conditions were as follows: an inlet temperature was 250° C., an initial temperature was maintained at 40° C. for 2 minutes, and temperature was raised to 290° C. at a rate of 20° C./min. MS conditions were as follows: ionization energy was 70 eV, an interface temperature was 280° C., an ion source temperature was 230° C., and a quadrupole temperature was 150° C. The control group did not include the cells, and the concentration of CF₄ thereof was 1,000 ppm. Then, incubation was performed under the same conditions, followed by measurement. The M9 medium includes 6 g of Na₂HPO₄, 3 g of KH₂PO₄, 0.5 g of NaCl, and 1 g of NH₄Cl per 1 L of distilled water.

Table 1 shows the amount of residual CF₄ after culturing BL21/pETD-dhIA, BL21/pETD-dhIA+pAD-RcIR, BL21/pETD-dhIA+pAD-RcIA, and BL21/pETD-dhIA+pAD-RcIABC for 4 days while in contact with CF₄ at a concentration of 1,000 ppm, relative to the control group.

TABLE 1

| | | Residual amount of | Reduction of |
|---|---|---|---|
| | | CF₄ (percentage (%) | CF₄ (percentage (%) |
| | | relative to the | relative to the |
| No. | Mutant | control group) | control group) |
| 1 | Control group | 100.00 | 0.00 |
| 2 | BL21/pETD-dhlA | 94.25 | 5.75 |
| 3 | BL21/pETD-dhlA + | 88.74 | 11.26 |
| | pAD-RclR | | |
| 4 | BL21/pETD-dhlA + | 87.84 | 12.16 |
| | pAD-RclA | | |
| 5 | BL21/pETD-dhlA + | 87.15 | 12.85 |
| | pAD-RclABC | | |

As shown in Table 1, the BL21/pETD-dhIA+pAD-RcIR, BL21/pETD-dhIA+pAD-RcIA, and BL21/pETD-dhIA+pAD-RcIABC strains respectively removed CF₄ in amounts 1.96 times, 2.11 times, and 2.23 times greater than the amount of CF₄ removed by the BL21/pETD-dhIA strain.

In addition, the BL21/pETD-dhIA, BL21/pETD-dhIA+pAD-RcIR, BL21/pETD-dhIA+pAD-RcIA, and BL21/pETD-dhIA+pAD-RcIABC E. coli strains were cultured in an LB medium while being stirred at 230 rpm and at a temperature of 30° C. At OD₆₀₀=0.5, 2.0 mM of IPTG was added thereto, and culturing was continued overnight with stirring at 230 rpm and at a temperature of 20° C. Next, the culture medium was centrifuged, and the supernatant was removed to obtain cells. A BugBuster protein extraction reagent (available from Novagen) was added to these cells, and the cell membranes were dissolved to thereby obtain a total cell lysate (T). The total cell lysate (T) was centrifuged. The supernatant was designated as a soluble fraction (S), and the sedimented pellet was designated as an insoluble fraction (IS). Sodium dodecylsulfate-polyacrylamide gel electrophoresis (SDS-PAGE) was performed on the total cell lysate (T), the soluble fraction (S), and the insoluble fraction (IS). The amount of expressed DhIA protein in each of T, S, and IS were compared with each other. As a control group, E. coli BL21 including an empty vector was used.

BL21/pETD-dhIA+pAD-RcIR, BL21/pETD-dhIA+pAD-RcIA, and BL21/pETD-dhIA+pAD-RcIABC, resulted in increased soluble expression of DhIA and decreased insoluble expression of DhIA, as compared with the control group and BL21/pETD-dhIA.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising a first genetic modification that increases activity of haloalkane dehalogenase (HAD) and a second genetic modification that increases expression of at least one gene selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4.

2. The recombinant microorganism of claim 1, wherein the first genetic modification increases the copy number of a gene encoding HAD, and the second genetic modification increases the copy number of at least one gene selected from the group consisting of the gene encoding an RcIR protein having about 95% or greater sequence identity to an amino acid sequence of SEQ ID NO: 1; the gene encoding an RcIA protein having about 95% or greater sequence identity to an amino acid sequence of SEQ ID NO: 2; the gene encoding an RcIB protein having about 95% or greater sequence identity to an amino acid sequence of SEQ ID NO: 3; and the gene encoding an RcIC protein having about 95% or greater sequence identity to an amino acid sequence of SEQ ID NO: 4.

3. The recombinant microorganism of claim 1, wherein the first genetic modification increases activity of HAD and the second genetic modification increases expression of the gene encoding an RcIR protein having about 95% or greater sequence identity to an amino acid sequence of SEQ ID NO: 1; or the first genetic modification increases activity of HAD and the second genetic modification increases expression of the gene encoding an RcIA protein having about 95% or greater sequence identity to an amino acid sequence of SEQ ID NO: 2; or the first genetic modification increases activity of HAD and the second genetic modification increases expression of the gene encoding an RcIA protein having about 95% or greater sequence identity to an amino acid sequence of SEQ ID NO: 2, and the gene encoding an RcIB protein having about 95% or greater sequence identity to an amino acid sequence of SEQ ID NO: 3, and the gene encoding an RcIC protein having about 95% or greater sequence identity to an amino acid sequence of SEQ ID NO: 4.

4. The recombinant microorganism of claim 1, wherein the HAD is classified as EC 3.8.1.5.

5. The recombinant microorganism of claim 1, wherein an ability of the recombinant microorganism to remove fluorinated methane in a sample is improved as compared with that of a parent strain of the recombinant microorganism, wherein the fluorinated methane is represented by CHₙF₄₋ₙ, wherein n is an integer from 0 to 3.

6. The recombinant microorganism of claim 1, wherein the gene encoding an RcIR protein, the gene encoding an RcIA protein, the gene encoding an RcIB protein, and the gene encoding an RcIC protein respectively have about 95% or greater sequence identity to nucleotide sequences of SEQ ID NOs: 5, 6, 7, and 8, respectively.

7. The recombinant microorganism of claim 1, wherein the recombinant microorganism belongs to the genus Escherichia.

8. The recombinant microorganism of claim 1, wherein the recombinant microorganism is E. coli.

9. A composition comprising a recombinant microorganism comprising a first genetic modification that increases activity of HAD and a second genetic modification that increases expression of at least one gene selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4 and a medium.

10. A method of reducing a concentration of fluorinated methane in a sample, the method comprising: contacting a recombinant microorganism with the sample comprising the fluorinated methane, wherein the recombinant microorganism comprises a first genetic modification that increases activity of HAD and a second genetic modification that increases expression of at least one gene selected from the group consisting of a gene encoding an RcIR protein having about 95% or greater sequence identity to an amino acid sequence of SEQ ID NO: 1; a gene encoding an RcIA protein having about 95% or greater sequence identity to an amino acid sequence of SEQ ID NO: 2; a gene encoding an RcIB protein having about 95% or greater sequence identity to an amino acid sequence of SEQ ID NO: 3; and a gene encoding an RcIC protein having about 95% or greater sequence identity to an amino acid sequence of SEQ ID NO: 4, wherein the fluorinated methane is represented by the formula CHₙF₄₋ₙ, and n is an integer from 0 to 3.

11. The method of claim 10, wherein the first genetic modification increases the copy number of a gene encoding HAD, and the second genetic modification increases the copy number of at least one gene selected from the group consisting of the gene encoding an RcIR protein having about 95% or greater sequence identity to an amino acid sequence of SEQ ID NO: 1; the gene encoding an RcIA protein having about 95% or greater sequence identity to an amino acid sequence of SEQ ID NO: 2; the gene encoding an RcIB protein having about 95% or greater sequence identity to an amino acid sequence of SEQ ID NO: 3; and the gene encoding an RcIC protein having about 95% or greater sequence identity to an amino acid sequence of SEQ ID NO: 4.

12. The method of claim 10, wherein the first genetic modification increases the activity of HAD and the second genetic modification increases the expression of the gene encoding an RcIR protein having about 95% or greater sequence identity to an amino acid sequence of SEQ ID NO: 1; or the first genetic modification increases activity of HAD and the second genetic modification increases expression of the gene encoding an RcIA protein having about 95% or greater sequence identity to an amino acid sequence of SEQ ID NO: 2; or the first genetic modification increases activity of HAD and the second genetic modification increases expression of the gene encoding an RcIA protein having about 95% or greater sequence identity to an amino acid sequence of SEQ ID NO: 2, and the gene encoding an RcIB protein having about 95% or greater sequence identity to an amino acid sequence of SEQ ID NO: 3, and the gene encoding an RcIC protein having about 95% or greater sequence identity to an amino acid sequence of SEQ ID NO: 4.

13. The method of claim 10, wherein the HAD is classified as EC 3.8.1.5.

14. The method of claim 10, wherein an ability of the recombinant microorganism to remove the fluorinated methane in the sample is improved as compared with that of a parent strain of the recombinant microorganism.

15. The method of claim 10, wherein the gene encoding an RcIR protein, the gene encoding an RcIA protein, the gene encoding an RcIB protein, and the gene encoding an RcIC protein respectively have about 95% or greater sequence identity to nucleotide sequences of SEQ ID NOs: 5, 6, 7, and 8, respectively.

16. The method of claim 10, wherein the recombinant microorganism belongs to a genus selected from the genus Escherichia, the genus Bacillus, and the genus Xanthobacter.

17. The method of claim 10, wherein the recombinant microorganism is E. coli.

18. The method of claim 10, wherein the contacting is performed in a sealed container.

19. The method of claim 10, wherein the contacting comprises culturing or incubating the recombinant microorganism while contacting the recombinant microorganism with the sample comprising fluorinated methane.

20. The method of claim 10, wherein the contacting comprises culturing the recombinant microorganism in a sealed container under conditions in which the recombinant microorganism is allowed to proliferate.

21. The recombinant microorganism of claim 1, wherein the HAD has an amino acid sequence having about 95% or greater sequence identity to an amino acid sequence of SEQ ID NO: 11.

22. The method of claim 10, wherein the HAD has an amino acid sequence having about 95% or greater sequence identity to an amino acid sequence of SEQ ID NO: 11.


SEQUENCE LISTING (16 sequences)

SEQ ID NO: 1 - protein, 284 aa, Escherichia coli
MDALSRLLMLNAPQGTIDKNCVLGSDWQLPHGAGELSVIRWHALTQGAAKLEMPTGEIFTLRPGNVVLLPQNSAHRLSHVDNESTCIVCGTLRLQHSARYFLTSLPETLFLAPVNHSVEYNWLREAIPFLQQESRSAMPGVDALCSQICATFFTLAVREWIAQVNTEKNILSLLLHPRLGAVIQQMLEMPGHAWTVESLASIAHMSRASFAQLFRDVSGTTPLAVLTKLRLQIAAQMFSRETLPVVVIAESVGYASESSFHKAFVREFGCTPGEYRERVRQLAP

SEQ ID NO: 2 - protein, 441 aa, Escherichia coli
MNKYQAVIIGFGKAGKTLAVTLAKAGWRVALIEQSNAMYGGTCINIGCIPTKTLVHDAQQHTDFVRAIQRKNEVVNFLRNKNFHNLADMPNIDVIDGQAEFINNHSLRVHRPEGNLEIHGEKIFINTGAQTVVPPIPGITTTPGVYDSTGLLNLKELPGHLGILGGGYIGVEFASMFANFGSKVTILEAASLFLPREDRDIADNIATILRDQGVDIILNAHVERISHHENQVQVHSEHAQLAVDALLIASGRQPATASLHPENAGIAVNERGATVVDKRLHTTADNIWAMGDVTGGLQFTYISLDDYRIVRDELLGEGKRSTDDRKNVPYSVFMTPPLSRVGMTEEQARESGADIQVVTLPVAAIPRARVMNDTRGVLKAIVDNKTQRMLGASLLCVDSHEMINIVKMVMDAGLPYSILRDQIFTHPSMSESLNDLFSLVK

SEQ ID NO: 3 - protein, 78 aa, Escherichia coli
MFKKSVLFATLLSGVMAFSTNADDKIILKHISVSSVSASPTVLEDAIADIARKYNASSWKVTSMRIDNNSTATAVLYK

SEQ ID NO: 4 - protein, 197 aa, Escherichia coli
MEKYLHLLSRGDKIGLTLIRLSIAIVFMWIGLLKFVPYEADSITPFVANSPLMSFFYEHPEDYKQYLTHEGEYKPEARAWQTANNTYGFSNGLGVVEVIIALLVLANPVNRWLGLLGGLMAFTTPLVTLSFLITTPEAWVPALGDAHHGFPYLSGAGRLVLKDTLMLAGAVMIMADSAREILKQRSNESSSTLKTEY

SEQ ID NO: 5 - DNA, 855 bp, Escherichia coli
atggatgcccttagccgtttgttgatgcttaacgctccacaaggaacgatcgataagaattgcgtgttaggaagtgactggcagcttccacatggtgccggggaattatcggttattcgttggcatgcgttaacgcaaggagcggcgaagctggaaatgccgacgggggagatttttacattacgcccgggaaatgtggtcctgctaccacaaaattcagctcatcgcctgagtcatgtcgataatgaatcgacctgtattgtctgcggcactcttcggttgcaacattcggcgcgttattttttaacgtctttgccggaaacgctgtttttagcaccggttaaccacagcgtggaatataactggctgagggaggcgatcccgtttttacaacaggaatccagatcggcaatgccgggagtggatgcactgtgtagccagatctgcgctacattctttaccctcgcggtgcgtgagtggattgcacaggttaatactgagaaaaacattctcagtttgcttctgcatccacgtcttggtgcggtaatacagcaaatgctggaaatgccaggacacgcctggaccgtcgaatcgctggccagcatcgctcacatgtcccgggcaagttttgcccagcttttccgtgatgtttccggaaccacgccgctggctgtattaacaaagttgcgtctacaaatagcggcccagatgttttcccgggaaacgctccctgttgtggtgatcgctgagtcagtaggctatgccagtgaatcatcttttcacaaggcgtttgtccgcgagtttggttgtaccccgggagaatatcgggaaagggtcagacagcttgcaccctga

SEQ ID NO: 6 - DNA, 1326 bp, Escherichia coli
atgaataaatatcaggcagtgattattggttttggcaaggctggaaaaacattagccgtcacgctggcaaaagcaggttggcgagtggctctcatcgaacaatcaaatgcaatgtatggcgggacctgtattaatatcggctgcatcccaaccaaaacattggttcatgacgcacagcagcacacagattttgtccgtgccatacagcgtaaaaatgaagtggttaattttttacgtaataagaattttcataatcttgcggatatgcccaatatcgacgtgatcgacggccaggcggagtttatcaataatcatagcctgcgtgttcatcggcctgagggaaatctggaaattcatggcgagaaaatttttattaataccggtgcacaaaccgtggttccgccaattcctggaattaccaccacgccaggagtatatgacagcaccggattacttaatctaaaagaattgcctgggcatttaggtattttgggcggcggatatattggcgttgagttcgcctctatgttcgctaattttggcagcaaggtaaccattttagaagcagcttcgctgtttttgcctcgggaagatcgggatattgctgataatatcgcgacgattttacgcgatcagggcgtcgatattatcctcaatgcccatgtggagcgaatcagtcaccatgaaaatcaagtgcaagtgcatagcgagcacgcccaactggcggtggatgcactgttaatagcttccggtcgtcaaccggctaccgcttcgttacatccagaaaatgccggtatcgcagtaaacgagcgcggggcaactgtcgttgacaagcgattacataccaccgcagacaatatttgggcgatgggagatgttaccggcgggctgcaatttacttacatatcactggatgattaccgcattgtacgtgatgagttactgggtgaaggcaaacgtagtactgatgatcggaaaaatgtgccttattccgtatttatgacaccgcccctgtccagggttggtatgacagaagaacaagccagagagagtggtgctgatattcaggtggtgacattgcctgtagctgcaattccgcgtgccagagtgatgaatgatactcgtggggtattaaaagcgattgttgataataaaacccaacgtatgttaggggcatcactgctgtgtgttgactcccacgagatgatcaatatagtgaaaatggtgatggatgccgggctgccttatagcatattacgcgatcagatatttactcatccgtcgatgagcgaatcactcaatgatctattttcattagtcaaataa

SEQ ID NO: 7 - DNA, 237 bp, Escherichia coli
atgtttaaaaaatccgttttatttgcaacgctattatctggcgttatggcattttccaccaatgcagatgataaaataattctgaaacatatcagcgtctcgtcagtatcagcatcaccgacagttctggaggatgccattgctgatatagccagaaaatataatgcttcatcctggaaagtcacatcgatgcgaattgataataattcaaccgcaacagcagtattgtataaataa

SEQ ID NO: 8 - DNA, 594 bp, Escherichia coli
atggaaaaatacctgcacctgttaagtcggggagataaaattggcctgacattgattcgtctgagtattgcaattgtttttatgtggattgggttattaaagtttgtcccttacgaggcagacagcattacaccattcgtcgcaaacagtccactaatgtcgttcttttatgaacacccggaagactataaacagtatctgactcacgaaggcgaatacaaaccagaagcaagggcatggcaaacggccaataatacctatggtttttccaacggtcttggcgtcgtggaggtgattattgctctgctggttttggctaatcctgtcaatcgctggttaggtttattgggaggactgatggcatttaccacgccgctggtaacactctcatttttaatcaccaccccggaggcatgggtacccgcattgggtgacgctcatcatggtttcccttatttatccggtgctggtcgcctggtattgaaagatactctgatgctggcaggtgctgtcatgataatggcagattcggcgcgggaaattcttaaacaacgcagtaatgaatccagttcaacgttaaaaactgaatattga

SEQ ID NO: 9 - DNA, 933 bp, Xanthobacter autotrophicus
  misc_feature (1)..(933): GJ10
atgataaatgcaattcgcaccccggaccaacgcttcagcaatctcgatcagtatccgttcagccccaactacctggacgacctccccggctacccgggattgcgggcacactacctcgacgagggcaattctgacgctgaagacgtttttctctgccttcatggcgagcccacctggagttacctgtatcgcaagatgatcccggtatttgctgaatcaggcgcacgagttattgcgccagacttttttggattcggaaaatccgacaagccagtagacgaagaagactacaccttcgaatttcaccgcaacttcctgcttgcactaatcgaacggcttgacttgcgcaacattacgctggtcgttcaggactggggcggatttttggggctgaccttaccgatggccgacccttcccgcttcaagcgcctgatcatcatgaacgcctgcttgatgaccgacccggtcacccagcctgcgtttagcgcctttgtcacccagcctgcggatggctttaccgcctggaaatacgatctggttacgccatcagacctgcgccttgaccagttcatgaagcgttgggcgcccacactgaccgaagctgaggcctccgcgtatgctgcgcctttccctgacacttcctatcaggctggtgtacgcaagtttcccaagatggtcgcgcaacgcgaccaggcctgcatcgacatttcaaccgaagcgatttcgttctggcagaacgactggaatggccagaccttcatggccattggcatgaaagacaaattgctgggaccggacgtcatgtatcctatgaaggcgctcattaatggctgcccggaacccctcgaaatagcggacgctggccatttcgtacaggagtttggcgagcaagtggctcgcgaggccctgaaacactttgccgagacagaatag

SEQ ID NO: 10 - DNA, 933 bp, Artificial Sequence
  Synthetic Escherichia coli codon optimized X. autotrophicus DhlA
atgatcaacgcaatccgcacccctgaccaacgtttcagcaacctggaccaataccctttcagccctaactacctggacgacctgccaggttacccaggtctgcgtgcacattacctggacgaaggtaacagcgacgctgaagacgttttcctgtgtctgcacggtgaaccaacttggagctacctgtaccgtaagatgatcccggtcttcgctgagtccggtgcacgtgttatcgcaccggatttcttcggtttcggtaagtccgacaagccggtagatgaggaggattacaccttcgagttccaccgtaacttcctgctggcgctgatcgaacgtctggatctgcgtaacatcaccctggtcgtgcaggattggggtggtttcctgggtctgactctgccgatggcagatccgtctcgttttaaacgtctgatcatcatgaacgcctgcctgatgaccgacccggtaactcagccggcattttccgcattcgttactcagccggcggatggctttactgcttggaaatatgacctggtgacgccgtctgatctgcgtctggatcagttcatgaaacgctgggctccgaccctgaccgaagctgaagcttctgcttatgctgcgccgtttccggatacctcttatcaggccggcgtacgcaaatttccgaaaatggtggcgcagcgcgatcaggcgtgtattgatatttctacggaagcgatctccttctggcagaacgactggaatggccagaccttcatggccattggcatgaaagacaaactgctgggcccggacgttatgtatccgatgaaagcgctgattaatggctgcccggaaccgctggaaattgcggacgccggccattttgtgcaggaattcggcgaacaggttgcgcgcgaagccctgaaacactttgcggaaaccgaataa

SEQ ID NO: 11 - protein, 310 aa, Xanthobacter autotrophicus
  misc_feature (1)..(310): GJ10
MINAIRTPDQRFSNLDQYPFSPNYLDDLPGYPGLRAHYLDEGNSDAEDVFLCLHGEPTWSYLYRKMIPVFAESGARVIAPDFFGFGKSDKPVDEEDYTFEFHRNFLLALIERLDLRNITLVVQDWGGFLGLTLPMADPSRFKRLIIMNACLMTDPVTQPAFSAFVTQPADGFTAWKYDLVTPSDLRLDQFMKRWAPTLTEAEASAYAAPFPDTSYQAGVRKFPKMVAQRDQACIDISTEAISFWQNDWNGQTFMAIGMKDKLLGPDVMYPMKALINGCPEPLEIADAGHFVQEFGEQVAREALKHFAETE

SEQ ID NO: 12 - DNA, 38 bp, Artificial Sequence
  Synthetic rclR_foward
taataaggagatataccatggatgcccttagccgtttg

SEQ ID NO: 13 - DNA, 38 bp, Artificial Sequence
  Synthetic rclR_reverse
cattatgcggccgcaagctttagggtgcaagctgtctg

SEQ ID NO: 14 - DNA, 38 bp, Artificial Sequence
  Synthetic rclA_foward
taataaggagatataccatgaataaatatcaggcagtg

SEQ ID NO: 15 - DNA, 40 bp, Artificial Sequence
  Synthetic rclA_reverse
cattatgcggccgcaagctttatttgactaatgaaaatag

SEQ ID NO: 16 - DNA, 39 bp, Artificial Sequence
  Synthetic rclABC_reverse
cattatgcggccgcaagctttaatattcagtttttaacg